(19) United States
(12) Patent Application Publication
(10) Pub. No.: US 2026/0022321 A1
(43) Pub. Date: Jan. 22, 2026

(54) SYSTEMS AND METHODS FOR INSTRUMENTED NEUROVASCULAR UNIT DEVICE

(71) Applicant: Lawrence Livermore National Security, LLC, Livermore, CA (US)

(72) Inventors: Nicholas FISCHER, Livermore, CA (US); Travis MASSEY, Livermore, CA (US); Heather Ann ENRIGHT, Livermore, CA (US); Noah Kaimana Takeyama GOSHI, Pleasanton, CA (US); Angela C. TOOKER, Tracy, CA (US); Michael Gregory TRIPLETT, Pleasanton, CA (US); Javier Antonio ALVARADO, Livermore, CA (US); Monica Lizet MOYA, Livermore, CA (US)

(21) Appl. No.: 18/774,634

(22) Filed: Jul. 16, 2024

(51) Int. Cl. C12M 1/34; C12M 1/00; C12M 1/32

(52) U.S. Cl. CPC C12M 41/36; C12M 23/12; C12M 23/38; C12M 29/16; C12M 41/12

(57) ABSTRACT

The present disclosure relates to a neurovascular cell culture system. The system may have an electrical/electronic interface board and a cell culture well secured to the interface board. A 3D microelectrode array is disposed within an interior volume of the cell culture well and in electrical communication with the interface board. The 3D microelectrode array has at least one probe with at least one electrode thereon. The cell culture well also has at least one fluidic port for enabling fluidic access to the interior volume of the cell culture well.

### FEDERALLY SPONSORED RESEARCH OR DEVELOPMENT

This invention was made with Government support under Contract No. DE-AC52-07NA27344 awarded by the United States Department of Energy. The Government has certain rights in the invention.

### FIELD

The present disclosure relates to systems and methods for forming neurovascular units, and more particularly neurovascular culture systems and methods having at least one cell culture well containing a 3D microelectrode array, and configured to permit fluidic access to an interior volume of the cell culture well.

### BACKGROUND

The statements in this section merely provide background information related to the present disclosure and may not constitute prior art.

The “blood-brain barrier” (“BBB”) is the term used to describe the semi-permeable barrier of the blood vessels that line the central nervous system (CNS), which tightly regulates the movement of cells, cytokines, small molecules, and ions between the CNS and periphery. This helps to maintain CNS homeostasis and neural function, while also protecting the brain from blood-borne pathogens and toxins. In combination with endothelial cells that form the direct barrier of the BBB, cells from the CNS including neurons, astrocytes, and pericytes form strong connections that regulate the function and permeability of the BBB. This functional unit of cells, deemed the neurovascular unit (NVU) is a critical component to maintaining neural function; dysregulation of the NVU is implicated in many neurological and neurodegenerative disorders including Parkinson's and Alzheimer's disease and amyotrophic lateral sclerosis. An effective in vitro model of the NVU that can both recapitulate and monitor physiologically relevant changes to both the BBB and brain function would provide a powerful platform to study neurological diseases, existing and emerging chemical and biological threats, and for development and evaluation of new brain therapeutics.

Current in vitro models of the BBB or NVU can largely be divided into three classes. The first involves culturing endothelial cells on a permeable support structure such as the commercially available Corning TRANSWELL® permeable support (i.e., insert), available from Cole-Palmer North America of Vernon Hills, IL. A TRANSWELL® insert is typically placed above a culture of neurons and/or glial cells. However, this configuration lacks the close proximity of the multiple cell types limiting its physiological relevance.

The second class of in vitro models of the BBB or NVU was pioneered by the Wyss Institute at Harvard University and commercialized by Emulate, Inc. This second class uses a microfluidic platform to culture endothelial cells and CNS cells near each other. In this platform, two microfluidic channels are separated by a porous polyethylene terephthalate (PET) film. Endothelial cells are cultured on one side of the PET film and neurons and glial cells are cultured on the opposing side of the film. While this allows for the direct contact between endothelial cells and CNS specific cells, this contact is limited to within the small pores in the PET film. Additionally, the CNS cells are cultured in 2D, limiting their physiological relevancy.

The third class of models involves culturing CNS cells within a hydrogel and culturing the endothelial cells along a surface or channel within the hydrogel. Typically, the hydrogel is formed within a millimeter scale channel and a secondary channel is formed within the hydrogel to simulate the blood vessel, which is lined with endothelial cells. This style of NVU model has significant benefits as the cells acquire the 3D configuration seen in vivo and there are no membranes separating the endothelial cells from the CNS cells. The main limitation with this third class of models is that it is difficult to maintain a healthy population of neurons within the hydrogel throughout the length of the experiment. There are two examples of NVU models with neurons present in the hydrogel. The first is from South Korea, which included secondary channels within the hydrogel to increase the exposure of neurons to fresh media. However, it is not clear if the neurons survived past five days in culture or showed any electrophysiological activity. A second group used a device setup similar to that commercialized by Mimetas Inc., in which three microfluidic “lanes” are positioned near each other. The center lane was filled with the hydrogel, with one adjoining lane lined with endothelial cells, and the opposing lane used to feed the neurons. This group was able to demonstrate neuron survivability and functional activity through 18 days in culture. However, this was done through studying the intracellular calcium transients and not through the direct recording of electrical activity. Furthermore, due to the device design, the hydrogel only interacted with a small fraction of the endothelial lined channel and no instrumentation (such as and MEA) was integrated into the CNS portion of the device.

### SUMMARY

This section provides a general summary of the disclosure, and is not a comprehensive disclosure of its full scope or all of its features.

In one aspect the present disclosure relates to a neurovascular cell culture system. The system may comprise an electrical/electronic interface board and a cell culture well secured to the interface board. The system may also include a 3D microelectrode array disposed within an interior volume of the cell culture well and in electrical communication with the interface board. The 3D microelectrode array has at least one probe with at least one electrode thereon. The cell culture well has at least one fluidic port for enabling fluidic access to the interior volume of the cell culture well.

In another aspect the present disclosure relates to a neurovascular cell culture assembly. The assembly may comprise a neurovascular cell culture system including an electrical/electronic interface board, a cell culture well secured to the interface board, and a 3D microelectrode array. The 3D microelectrode array is disposed within an interior volume of the cell culture well and in electrical communication with the interface board. The 3D microelectrode array has at least one probe with at least one electrode thereon, and the cell culture well has at least one fluidic port for enabling fluidic access to the interior volume of the cell culture well. The system may also include an enclosure assembly for containing the neurovascular cell culture system in a temperature controlled environment.

In still another aspect the present disclosure relates to a method for forming a monitoring neurovascular cell formation. The method may comprise using an electrical/electronic interface board to support a cell culture well thereon, and arranging a 3D microelectrode array within an interior volume of the cell culture well, and such that the 3D microelectrode is in electrical communication with the interface board. The 3D microelectrode array may have at least one probe with at least one electrode thereon. The method may further comprise at least partially filling an interior volume of the cell culture well with a substance which encases the 3D microelectrode array. The method may further include providing fluidic access to the 3D microelectrode array. The method may further include using an electronic subsystem to communicate with the 3D microelectrode array to at least one of: obtain electrical signals collected by the 3D microelectrode array, or provide electrical signals to the 3D microelectrode array.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

Corresponding reference numerals indicate corresponding parts throughout the several views of the drawings.

FIG. 1 is a highly enlarged perspective view of an in vitro cell culture system in accordance with one embodiment of the present disclosure;

FIG. 2 is a highly enlarged perspective view of one of the cells of the system shown in FIG. 1;

FIG. 3 is a highly enlarged perspective view of one of the cells of FIG. 1 with the inset figure showing in even further enlarged fashion the probes of one of the 3DMEA assemblies;

FIG. 4 is a cross-sectional side view of the cell 18a taken in accordance with section line 4-4 in FIG. 2 better illustrating the two tubes which enable fluidic access to the interior of the cell, as well as the cross-sectional construction of the well;

FIG. 5 is a perspective, transparent view of the cell of FIG. 1 further illustrating the construction of cap and the well;

FIG. 6 is a highly enlarged plan view of the probes of the 3DMEA showing the probes in their “unactuated (i.e., flat) orientation, before being actuated (i.e., formed into their upstanding, final orientation in FIG. 7);

FIG. 7 is a highly enlarged perspective plan view of the 3DMEA of FIG. 6 but with the probes in their fully actuated orientation (i.e., final, upstanding orientation);

FIG. 8 is a perspective view of one embodiment of an enclosure which may be used to house the cell culture system of FIG. 1, together with an amplifier interfaced thereto;

FIG. 9a is a side cross-sectional view of the enclosure of FIG. 8 along directional arrow 9a in FIG. 8 showing the fluid inlet and outlet which communicate with the tubes of each cell, as well as the use of an EMI/RF gasket to help promote EMI shielding for the enclosure;

FIG. 9b is a side cross-sectional view of the enclosure taken along direction line 9b in FIG. 8, further illustrating the construction of the enclosure; and

FIG. 10 is a highly enlarged, perspective, exploded view of the base plate and other components forming the enclosure assembly of FIGS. 8, 9a and 9b.

### DETAILED DESCRIPTION

Example embodiments will now be described more fully with reference to the accompanying drawings.

The systems and methods of the present disclosure overcome many limitations found in the prior art. For one, an NVU of the present disclosure 1) supports neuronal co-cultures in a hydrogel directly in contact with endothelial cells deposited on the inner surface of a microfluidic channel formed through the hydrogel; 2) allows for static or continuous flow through the vascular channel; 3) incorporates 3D multielectrode arrays to non-invasively interrogate the neuronal coculture; and 4) interfaces with commercial electrophysiology instrumentation.

The systems and methods described herein relate to an in vitro cell culture system consisting of at least one cell culture well containing a 3DMEA fabricated from flexible polymeric probes with one or more metal electrodes along each probe. The electrodes are capable of electrophysiological recording, stimulation, and can be further functionalized with additional coatings for the detection of chemical compounds. The cell culture well surrounds one or more spaced apart probe arrays and incorporates fluidic ports in communication with the well to enable direct fluidic access to the cell culture space (i.e., volume) within the well. Optionally, a cap can be attached to the culture well to create a closed system capable of dynamic cell culture conditions. The culture well cap also contains fluidic ports to enable independent fluidic access to the culture head space.

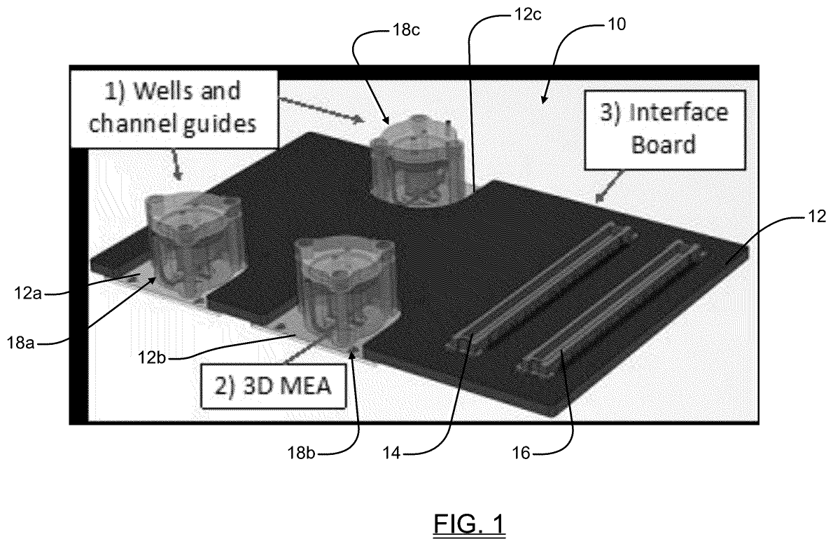

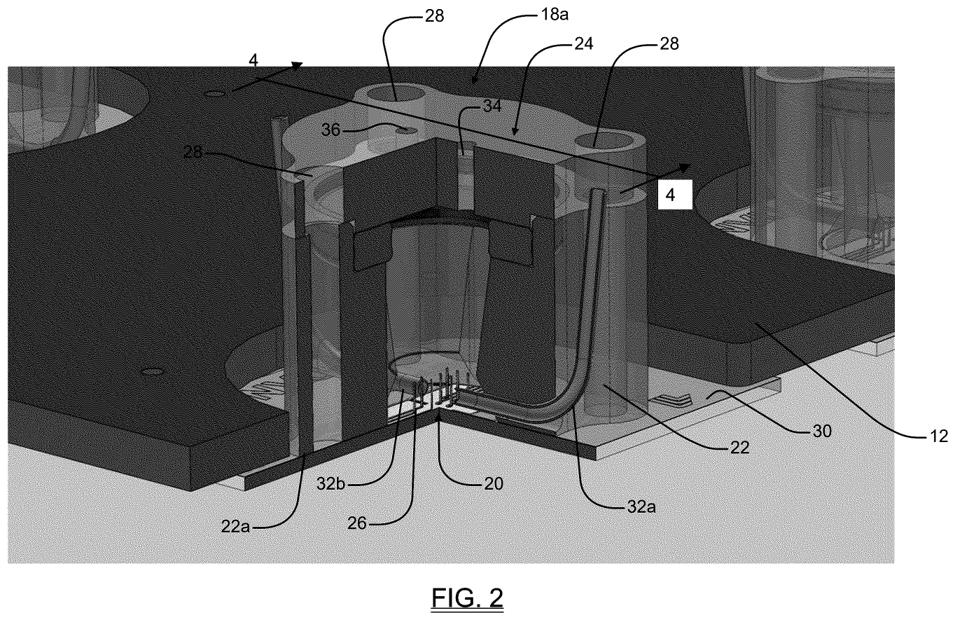

Referring to FIG. 1, one embodiment of an in vitro cell culture system 10 as outlined above is shown in accordance with the present disclosure. The in vitro cell culture system 10 (hereinafter simply “system 10”) in this example includes a microfabricated electrical/electronic interface board 12 having a plurality of electrical connectors 14 and 16 and printed circuit traces (not clearly visible) for electrically coupling to external electronics equipment. The interface board 12 in this embodiment includes three recessed areas 12a, 12b and 12c where corresponding in vitro cells 18a, 18b and 18c are located. FIG. 2 shows the cell 18a in greater detail. Since the cells 18a, 18b and 18c are all identical in construction in this example (although they need not be), only cell 18a will be described in detail. It will be appreciated that a greater or lesser number of cell 18 may be included to meet the needs of a specific application.

With reference FIG. 2, cell 18a can be seen to include 3DMEA (3D microelectrode array) 20 enclosed within a well 22. The well 22 in this embodiment is circular, although it may take other cross-sectional shapes (e.g., square or rectangular), and has a removable cap 24 which encloses an upper edge of the well. In some embodiments the well 22 may be made from a polymer, for example polystyrene, or from glass, metal or any other suitable material. The well 22 may be transparent or opaque, and may be 3D printed, molded or formed in any other suitable manner. The 3DMEA 20 in this example has ten independent probes 26 each having a plurality of independent electrodes 26a, although a greater or less number of probes 26 and/or electrodes 26a could be included to meet a specific application. Recesses 28 form locations where threaded elements such as threaded fasteners may be inserted through holes (not visible) in the cap 24 and into threaded bores in an upper edge of the well 22 to secure the cap to an upper surface of the well. A lower edge 22a of the well 22 may be secured to a microfabricated chip structure 30, which forms a portion of the interface board 12, and which contains the 3DMEAs 20 thereon. Securing may be accomplished via any suitable means, for example and without limitation, by suitable adhesives. One suitable adhesive is EPO-TEK® MED-353ND adhesive available from Epoxy Technology of Billerica, MA.

In some embodiments the microfabricated chip structure 30 which contains the 3DMEA 20 thereon may form a footprint of about 14.5 mm×20.5 mm and be electrically connected to the interface board 12 via any suitable means, for example and without limitation, by solder reflow or conductive epoxy. In some embodiments the three wells 18a, 18b and 18c provide 80 stimulation/recording channels for each well 18a, 18b and 18c. Overall, in some embodiments the system 10 may have a footprint of about 2″×3″, which is a common size for microscope slides, and which significantly simplifies imaging and handling.

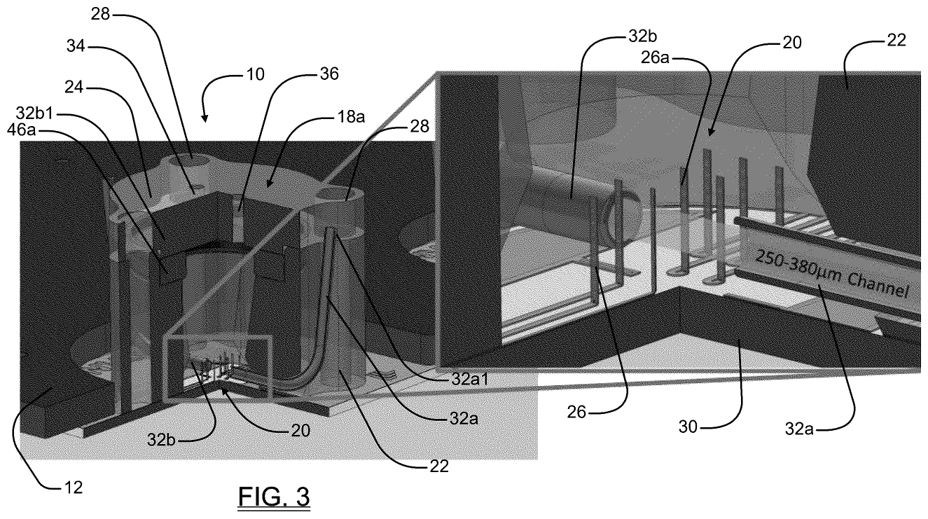

With further reference to FIGS. 2-4, the well 22 may also include a pair of independent flow tubes 32a and 32b which may be inserted through openings 38 and 40, respectively, into an interior volume of the well 22. Distal ends of the tubes 32a and 32b are arranged in facing relationship adjacent to the probes 26 of the 3DMEA 20 (FIG. 3) to form a channel through which fluidic access to the interior volume of the 18a can be gained. The tubes 32a/32b themselves may be made from any suitable material, but in some embodiments the tubes are formed from stainless steel hypodermic tubing and pre-bent into the shapes shown in FIG. 4 before being inserted through the openings 38 and 40 in the well 22. Suitable adhesives, such as the EPO-TEK adhesive described above, may be used to secure and seal passageways 38 and 40 through which the tubes 32a and 32b enter the well 22. The upper ends of the flow tubes 32a and 32b, as shown in FIG. 4, form communication ports 32a1 and 32b1 through which fluidic access to the region of the 3DMEA 20 is gained. The interior diameter of the tubes 32a/32b may vary significantly in diameter, although it is anticipated that in most applications an inner diameter from about 250 μm-400 μm will be acceptable.

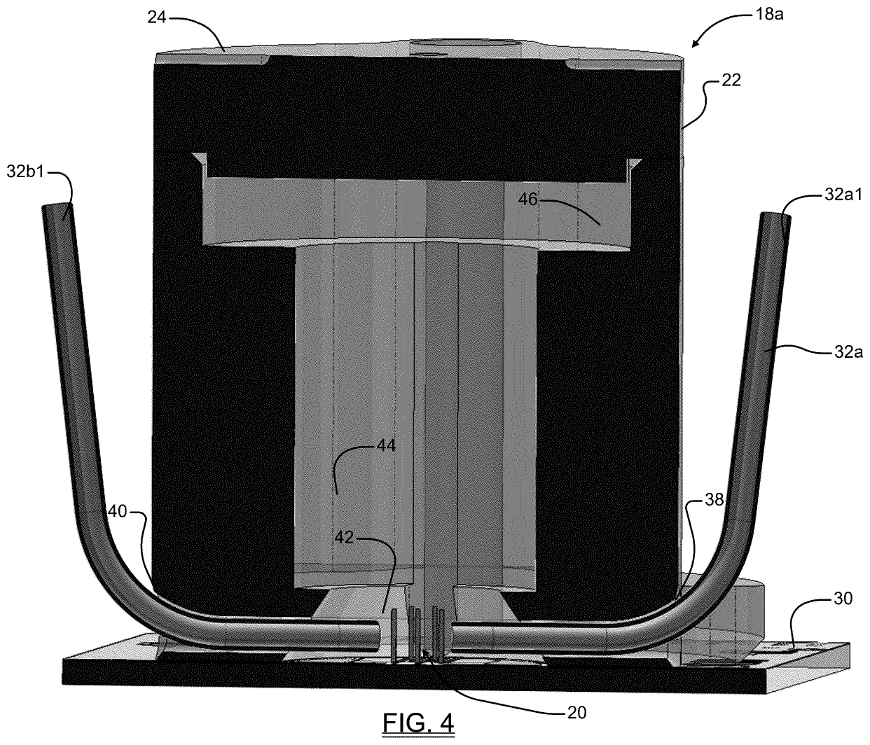

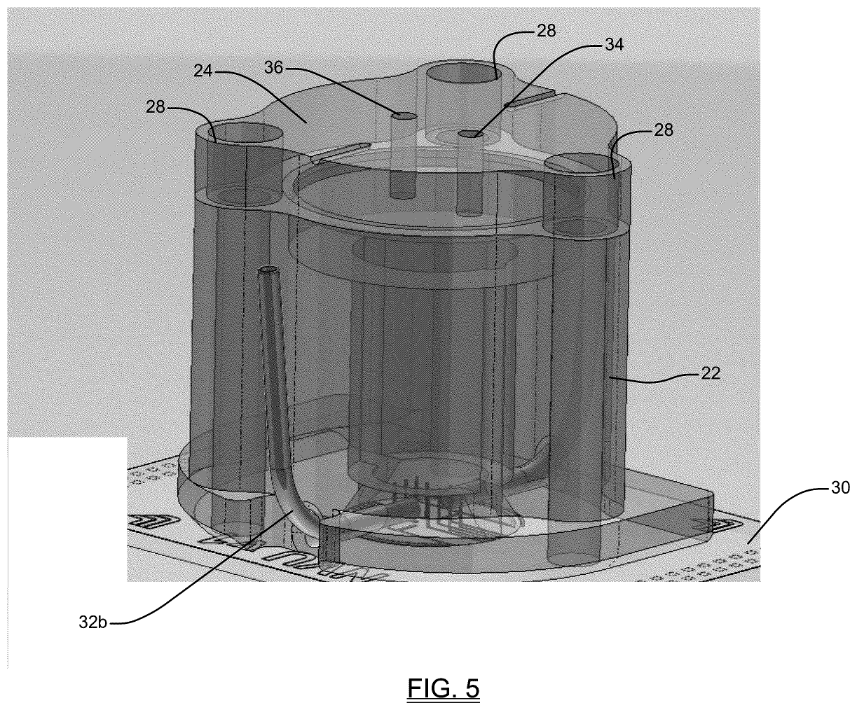

With further reference to FIGS. 3 and 5, the well cap 24 may also include an additional pair of openings 34 and 36 in the cap 24 to gain access to a headspace above the media (e.g., hydrogel) contained in the well 22. The openings 32a1 and 32b1 enable a perfusable lumen to be formed in the media contained within the cell 18a, which in one example may be a hydrogel. This may be accomplished by inserting a metal pin or length of wire though one of the openings 32a1 or 32b1 and into the hydrogel prior to a polymerization process being performed. In this example, once the hydrogel is polymerized, the pin/wire can be removed, leaving a hollow tubular channel in the hydrogel that can be fluidically accessed using the integrated fluidic ports 32a1 and 32b1 and the flow tubes 32a/32b, respectively, which are in communication with the interior volume of the cell 18a. This hollow channel enables the introduction of media, chemical challenges, biological challenges, etc. directly to the interior of the 3D gel within the cell 18a. This hollow channel may also be coated with alternate cell types (e.g., endothelial cells, pericytes) to model complex integrated biological systems (e.g., neurovascular unit). Cells coating the lumen walls can be cultured under dynamic conditions by continuously circulating media through the lumen via the integrated fluidic ports on the well. Depending on the desired culture conditions, the top of the well 22 can be sealed using the cap 24. The fluidic ports 34 and 36 enable access to the headspace above the hydrogel without removal of the cap for fluid introduction or removal.

With further brief reference to FIG. 4, a highly enlarged, side cross section view of the well 22 can be seen. The 3DMEA is disposed in a lower region 42 of the well 22. A mid-region 44 of substantially larger volume than the lower region 42 exists above the lower region, and a O-ring groove 46 is present above the mid-region 44. The lower region 42 will typically be filled with a substance, in some embodiments a polymerized hydrogel, before the system 10 is put into use. In some embodiments dissociated electroactive cells (e.g., neuronal cells, cardiac cells) may be mixed with the hydrogel before the hydrogel is polymerized to form the solidified hydrogel in which the 3D distribution of cells is maintained. As the distributed cells mature over days or weeks, networks are allowed to form around the electrodes 26a of the probes 26, and the resulting electroactive function can be measured and recorded using the 3DMEA 20 and compatible instrumentation. The headspace 44 may be used to hold cell culture media, which can be supplemented (e.g., nutrients, vitamins, or growth factors) as required for promoting the health and function of the cells composing the neurovascular unit, or other imaging and assay buffers to interrogate the cells within the device. The headspace 44 may also be used to introduce biological or chemical elements (e.g., inhibitory or excitatory molecules or pathogens) to modulate or validate the function of the cellular constituents of the neurovascular unit. The O-ring groove 46 provides space for an O-ring 46a (shown in FIG. 3)) to generate a liquid tight seal between the cap 24 and the well 22.

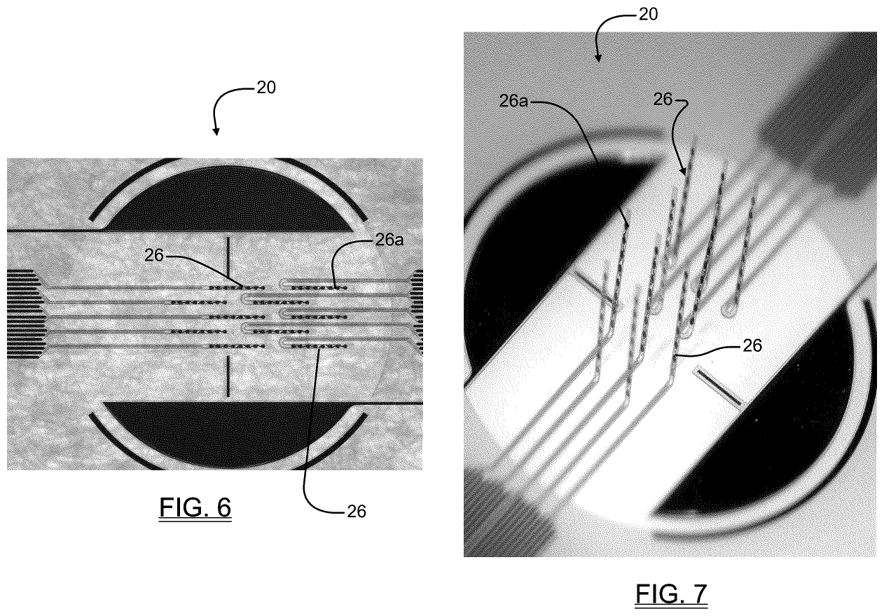

Referring to FIGS. 6 and 7, the 3DMEA 20 can be seen before the probes 26 have been bent into their upright extending configurations (FIG. 6) and in their final, upright configurations. Specific details on how the probes 26 can be bent from the flat configuration shown in FIG. 6 to the final, upright configuration shown in FIG. 7 may be found in U.S. Pat. No. 11,725,170 to Soscia et al., issued Aug. 15, 2023 and assigned to the assignee to Lawrence Livermore National Security, LLC., the full disclosure of which is hereby incorporated by reference. While FIGS. 6 and 7 show the 3DMEA 20 with ten probes 26, it will be appreciated that the present disclosure is not limited to the use of only ten probes, and the specific number, as well as the dimensions (e.g., length and width), shape and spacing between the probes may depend on the specific application that the system 10 is being used with and other factors. Cellular complexes may also be formed around the actuated probes 26 using spheroids or organoids consisting of electroactive cells. The stiffness of the probes 26 can be modulated if necessary to enable penetration of a pre-formed organoid or cell-hydrogel construct to record or stimulate inside of the volume of the cell 20 (e.g., inside the lower third region 42 shown in FIG. 4).

The 3DMEAs 20 may be fabricated using wafer-level fabrication which allows for easy customization of probe 26 length, width, thickness, number, position, probe body material, and probe material to fit specific applications. Probe 26 bodies may be fabricated using any patternable, flexible, biocompatible polymer such as polyimide, silicones, and parylene. The metals used for the probe electrodes 26a can be fabricated using biocompatible metals such as gold, platinum, titanium, and iridium or a combination of multiple metals. The electrode 26a surfaces can be further coated with other metals, polymers, or biomolecules to improve performance, biocompatibility, or functionality of the probes 26.

The 3DMEA 20 form factor may also be customized as necessary to better ensure compatibility and fit with specific desired applications. While the 3DMEAs 20 may be fabricated to directly interface with compatible instrumentation for electrical recording and or stimulation, the 3DMEAs 20 as described herein may also be electrically connected to a larger interposer circuit board which connects directly to compatible instrumentation. This enables potential increases in 3DMEA yield and fabrication throughput by reducing footprint of microfabricated components and facilitating device modularity (e.g., the same 3DMEA design can be used in different measurement systems or in different quantities on the same measurement system by using a different interposer board). In some embodiments, as fabricated, the 3DMEA 20 consists of a 2D planar field of one or more of the probes 26. In some embodiments, such as shown in FIGS. 5 and 6, and as described also in U.S. Pat. No. 11,725,170, each probe 26 consists of a main probe body that is detached from the 3DMEA substrate (e.g., portion 30 of interface board 12), an adjacent hinge region that may be fully or partially released from the surface, and a third region that is permanently affixed to the surface of the 3DMEA 20. The main probe 26 body can be actuated to a vertical or diagonal position relative to the base substrate. The hinge region may include a void in the polymer substrate to encourage preferential bending in that region. Plastic deformation of the polymer in the hinge region during actuation allows the probes 26 to remain in the actuated position without any additional fixing elements or external force after the actuation process. Actuation is achieved using a process of controlled buckling and lifting of the probes from the 2D configuration, as described in U.S. Pat. No. 11,725,170. Buckling shanks are positioned at the distal end of the probe 26 and a compressive force may be applied tangent to the MEA surface in the direction of the probe hinge. The compressive force causes the main body of the probe 26 to buckle and form an arch above the MEA surface. Lifting shanks may be positioned perpendicular to the probe body, as explained in U.S. Pat. No. 11,715,170, and moved underneath the arched probes. Once underneath the arched probes the lifting shanks are moved towards the probe hinge to lift the probes 26 into the actuated position. In this manner, simultaneous actuation of multiple probes is achieved using microfabricated buckling/lifting shanks and an actuation apparatus consisting of multiple micromanipulators. This enables precise positioning of the microfabricated buckling/lifting shanks, and the use of two positioning cameras enables visualization of the actuation shanks and probes during the actuation process. Once the probes 26 are actuated, the well 22 can be bonded to the 3DMEA substrate (e.g., component 30 of the interface board 12) surrounding one or more 3DMEA probe arrays 20.

Referring to FIGS. 8, 9a, 9b and 10, one example of how the system 10 may be integrated with additional electronic components and subsystems is shown. In FIG. 8 a base plate 100, which in one embodiment is of metal construction and magnetic, is used to house the system 10. A lid 102, which in one embodiment is partially of metal construction and magnetic, may be used to fully enclose the system 10 by interfacing to an upper edge surface of the base plate 100. The base plate 100 and lid 102 effectively form a small Faraday cage that shields the system 10 from noise, which otherwise could make difficult or impossible to measure the very low magnitude electrical signals detected by the probe electrodes 26a. The enclosure formed by the base plate 100 and the lid 102 also helps to maintain specific gaseous and thermal environments necessary to ensure that the system 10 experiences consistent and controlled environmental parameters, while the magnetic attachment of the lid 102 enables quick and easy, tool-free access to the system 10 when needed.

With further reference to FIG. 8, the base plate 100 may be interfaced to other electronic equipment, for example an amplifier 106, which is electrically interfaced to the interface board 12 via electrical connectors 14 and 16 and suitable cabling or electrical connectors (not shown, for example ribbon cables). In some embodiments the amplifier 106 may be a linear amplifier commercially available from Harvard Bioscience, Inc. of Holliston, MA.

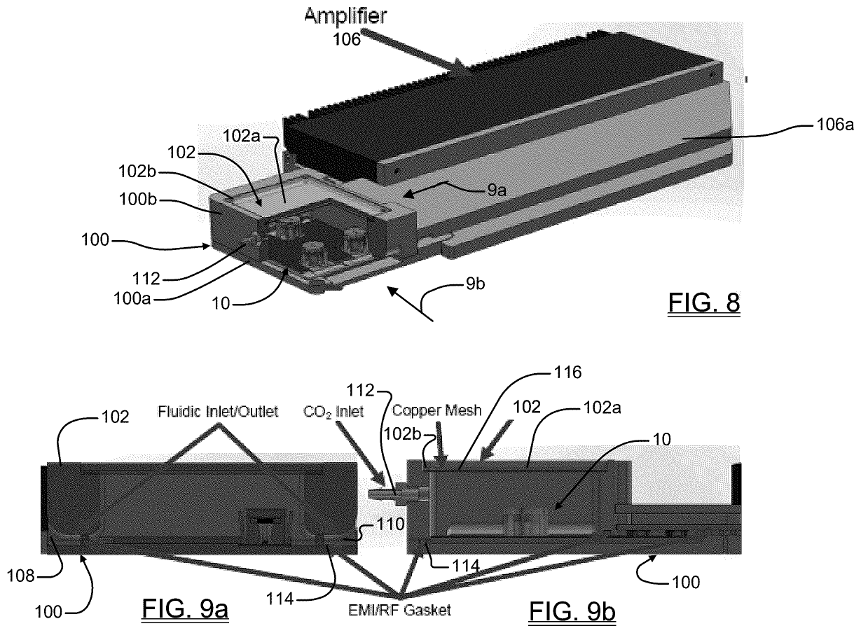

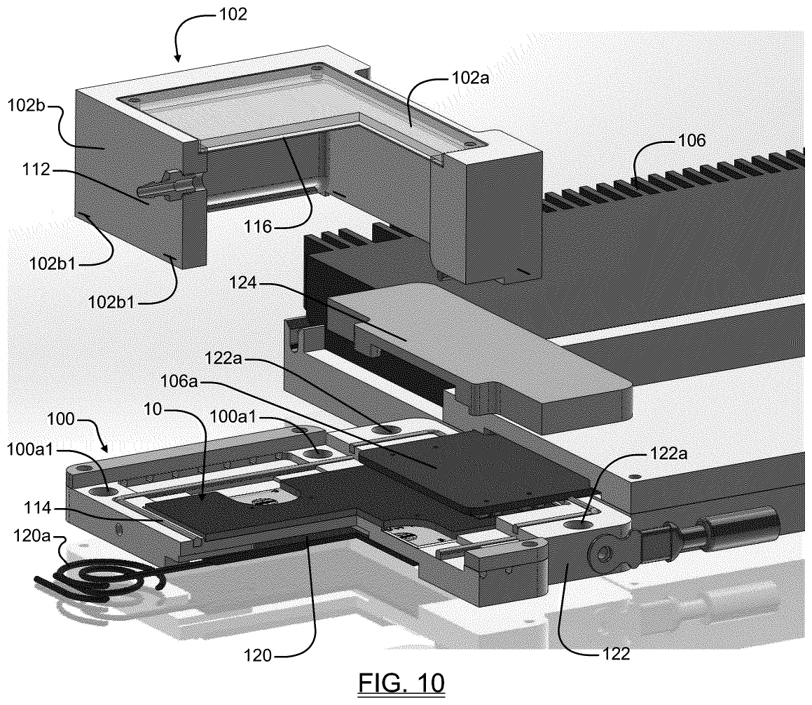

FIGS. 8, 9a, and 9b show that the base plate 100 may also include ports for the fluidic inlet tubing 108 and a fluidic outlet tubing 110, which communicate with the tubes 32a and 32b of each cell 18a, 18b and 18c to pass under the lid 102 and into the enclosure created by the base plate assembly 100 and the lid 102. This permits fluids to be exchanged within the cells 18a, 18b and 18c of the system 10 without the need to remove the system 10 from the enclosure formed by the base plate 100 and the lid 102. A gas inlet port 112 may also be included on the lid assembly 102 to enable the introduction and containment of gases (e.g., and without limitation, air with specific CO₂ percentages) within the base plate 100 when it is closed off. An EMI/RF gasket 114 may be included to help provide electrical shielding between the base plate 100 and the lid assembly 102.

As shown in FIGS. 9a, 9b and 10, the lid 102 may include an acrylic panel portion 102a forming a window, a metal frame 102b, and a copper mesh 116 layer may also be provided on an undersurface of the acrylic panel portion 102a forming the window (FIG. 9b) to maintain electrical shielding of the system 10 from noise while enabling users to see the system 10 without removing the lid 102. In some embodiments 5% CO₂ and 37° C. is maintained within the base plate 100 during use of the system 10. Preferably, a small planar (e.g., 20 W) silicone heater 120, visible in FIG. 10, is also included within the base plate 100 to help maintain the desired temperature within the enclosure created by the base plate 100 and the lid 102. An electrical cable 120a associated with the heater 120, enables DC power to be provided to the heater from an external power source during use of the system 10. FIG. 10 also illustrates that the EMI/RF gasket 114 extends within a groove in a base structure 122 which supports the amplifier 106. FIG. 10 also shows a portion of a circuit board 106a which is coupled to the connectors 14 and 16 (only visible in FIG. 1). This portion of the circuit board 106 is enclosed by a releasably secured metal cover 124. The cover 124 may be secured by any conventional means, but in this example magnets are positioned in bores 122a and the metal cover 124 is magnetically attached thereto, enabling easy removal by hand without external tools.

From the above, and with specific reference to FIG. 10, it will be appreciated then that the lid 102, in some embodiments, may consist of three components: the metal frame 102b with four embedded magnets (shown in simplified form and identified by reference number 102b1), the copper mesh 116 which is in electrical contact with the metal frame, and the acrylic window 102a. The acrylic window 102a plus the copper mesh 116 may be bolted down (or otherwise suitably secured) to the metal frame 102b. In embodiments the acrylic window 116 (which could be formed by any transparent polymer) prevents gas/heat loss through the lid 102 and the copper mesh 116 acts to reduce noise. The lid 102 could be of full metal construction (i.e., without the copper mesh/acrylic window), but the user would need to remove the lid to see inside each cell 18. The base plate 100 has pockets milled into a surface that the system 10 sits in. Embedded magnets (removed for clarity) may be positioned in four bores 100a1 in the upper edge surface of the base plate 100 (only two of the bores 100a1 being visible) to interact with the embedded magnets 102b1 in the lid 102 frame 102b to hold the two pieces releasably together. There may also be pockets milled in the lid 102 and base plate 100 to allow fluidic tubing to pass between the lid and the base plate 100 and connect to fluidic ports 32a, 32b, 34, and 36 on the system 10. EMI/RF gasket material 114 can thus be installed on the base plate 100 and on the lid 102 to ensure they are electrically connected. If the base plate 100 has the planar silicone heater 120 installed, then this will enable the base plate to be heated to a desired temperature (for example, in this case 37C, to mimic the incubator).

The various embodiments of the system 10 and methods described herein are expected to find significant utility Research involving neuronal communication in 3D, blood brain barrier dysfunction, drug development, drug permeability, countermeasure validation, neuroinflammation, pathogen infection, chemical exposure, response of electroactive cells in 3D, disease research (Alzheimer's, Parkinson's, epilepsy, TBI, ALS etc.), recording/stimulation of spheroids/organoids, microfluidic delivery of chemicals in 3D.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items. As used herein, the term “about”, when used immediately previous to a specific recited value, denotes the specific recited value as well as all values, inclusive, from +/−10% of the specific recited value.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A neurovascular cell culture system comprising:
an electrical/electronic interface board;
a cell culture well secured to the interface board;
a 3D microelectrode array disposed within an interior volume of the cell culture well and in electrical communication with the interface board, the 3D microelectrode array having at least one probe with at least one electrode thereon; and
the cell culture well having at least one fluidic port for enabling fluidic access to the interior volume of the cell culture well.

2. The system of claim 1, wherein the at least one probe comprises a plurality of spaced apart, independent probes, with each one of the plurality of spaced apart, independent probes including a plurality of electrodes formed thereon.

3. The system of claim 1, wherein the at least one fluidic port is formed by a tube communicating with a region within the cell culture well adjacent to the 3D microelectrode.

4. The system of claim 1, wherein the at least one fluidic port comprises two independent tubes extending through openings in the cell culture well, with each said tube being in communication with a region adjacent to the 3D microelectrode within the volume of the cell culture well.

5. The system of claim 1, further comprising a cap for covering an upper area of the cell culture well.

6. The system of claim 1, wherein the cap includes at least one opening for enabling communication with the interior area of the well.

7. The system of claim 1, wherein the well includes:
a lower region within which the 3D microelectrode is disposed;
an intermediate region elevationally above the lower region forming a headspace, and which is substantially larger in volume than the lower region; and
a region elevationally above the intermediate for receiving an O-ring.

8. The system of claim 1, wherein the well includes a polymerized hydrogel encasing the 3D microelectrode.

9. The system of claim 8, wherein:
the well comprises:
a lower region within which the 3D microelectrode is disposed;
an intermediate region elevationally above the lower region which is substantially larger in volume than the lower region and which forms a headspace; and
an O-ring groove above the intermediate region; and
a polymerized hydrogel encasing the 3D microelectrode.

10. The system of claim 1, wherein the probe is configured to extend substantially perpendicularly to the interface board.

11. A neurovascular cell culture assembly, comprising:
a neurovascular cell culture system including:
an electrical/electronic interface board;
a cell culture well secured to the interface board;
a 3D microelectrode array disposed within an interior volume of the cell culture well and in electrical communication with the interface board, the 3D microelectrode array having at least one probe with at least one electrode thereon; and
the cell culture well having at least one fluidic port for enabling fluidic access to the interior volume of the cell culture well; and
an enclosure assembly for containing the neurovascular cell culture system in a temperature controlled environment.

12. The assembly of claim 11, further comprising an amplifier in electrical communication with the neurovascular cell culture system.

13. The assembly of claim 11, wherein the cell culture well comprises a plurality of independent cell culture wells secured to the interface board.

14. The assembly of claim 11, further comprising at least one tube extending through an opening in the cell culture well into an area adjacent to the 3D microelectrode, to provide fluidic access to the interior volume of the cell culture well.

15. The assembly of claim 14, wherein the at least one tube comprises two independent tubes extending through a pair of openings in the cell culture wall into the interior volume of the cell culture well, and each said tube including a distal end being disposed adjacent to the 3D microelectrode.

16. The assembly of claim 11, wherein the assembly includes a cap for covering an uppermost area of the cell culture well.

17. The assembly of claim 16, wherein the cap is removably coupled to the cell culture well and includes at least one opening for permitting access to the interior volume of the cell culture well when the cap is secured to the cell culture well.

18. The assembly of claim 11, wherein the at least one probe comprises a plurality of independent, spaced apart probes each having a plurality of electrodes formed thereon.

19. The assembly of claim 18, wherein the plurality of independent, spaced apart probes are configured to extend generally perpendicularly relative to the electrical/electronics interface board within the cell culture well.

20. A method for forming a monitoring neurovascular cell formation, comprising:
using an electrical/electronic interface board to support a cell culture well thereon;
arranging a 3D microelectrode array within an interior volume of the cell culture well, and such that the 3D microelectrode is in electrical communication with the interface board, wherein the 3D microelectrode array has at least one probe with at least one electrode thereon;
at least partially filling an interior volume of the cell culture well with a substance which encases the 3D microelectrode array;
providing fluidic access to the 3D microelectrode array; and
using an electronic subsystem to communicate with the 3D microelectrode array to at least one of:
obtain electrical signals collected by the 3D microelectrode array, or
provide electrical signals to the 3D microelectrode array.
